(19) United States
(12) Patent Application Publication
(10) Pub. No.: US 2024/0335217 A1
(43) Pub. Date: Oct. 10, 2024

(54) ANIMAL (NON-HUMAN) ILIAL FRACTURE STABILIZATION

(71) Applicant: Samuel Patrick Franklin, Overland Park, KS (US)

(72) Inventor: Samuel Patrick Franklin, Overland Park, KS (US)

(21) Appl. No.: 18/294,863

(22) PCT Filed: Aug. 2, 2022
(86) PCT No.: PCT/US2022/039214

§ 371 Date: Feb. 2, 2024

Related U.S. Application Data
(60) Provisional application No. 63/228,879, filed on Aug. 3, 2021.

(51) Int. Cl. A61B 17/80

(52) U.S. Cl. CPC A61B 17/8066; A61B 17/8052

(57) ABSTRACT

Aspects of the present disclosure provide fracture plates for stabilizing fractures of the ilium in canines and other animals. Some of the fracture plates include multiple rows of screw holes configured to receive locking or non-locking screws. The screw holes are positioned to facilitate attachment of the fracture plate to the bone with multiple screws on both sides of the fracture, in stronger portions of the ilium.

### CROSS-REFERENCE TO RELATED APPLICATIONS

This application claims the benefit under 35 U.S.C. § 119 (e) of U.S. Provisional Patent Application Ser. No. 63/228,879, filed Aug. 3, 2021 and entitled “Animal (Non-Human) Ilial Fracture Stabilization,” which is hereby incorporated by reference herein in its entirety.

### FIELD

The present disclosure relates to methods and apparatuses for stabilization of fractures of the ilium in non-human animals.

### BACKGROUND

Fractures of the ilium occur in canines, felines, and other animals. They are sometimes treated by securing a bone plate to the ilium. Typically, the bone plate has a single, linear row of screw holes.

### SUMMARY

Apparatus and methods for ilial fracture stabilization are provided.

According to an aspect of the present disclosure, an apparatus for stabilization of ilial fractures is provided. The apparatus comprises a planar or curved fracture plate having a first end and second end, and two rows of screw holes positioned between the first end and second end. Each of the two rows of screw holes comprises three or more screw holes. A total of between six and twelve screw holes is provided. The planar or curved fracture plate has a length, a width, and a thickness. The width is greater than a combined width of two adjacent screw holes along a dimension of the width. The width is variable along the length such that the width is narrower at the first end than at a midpoint of the planar or curved fracture plate.

According to an aspect of the present disclosure, an apparatus for stabilization of ilial fractures is provided. The apparatus comprises a planar or curved fracture plate comprising multiple rows of screw holes. The planar or curved fracture plate has a length, a width, and a thickness. The width of the fracture plate is greater than a combined opening size of two screw holes along a dimension of the width.

According to an aspect of the present disclosure, a method for stabilizing a fracture of the ilium is provided. The method comprises: positioning a single fracture plate on a lateral surface of the ilium straddling the fracture such that screw holes in two substantially parallel rows are on either side of the fracture; and screwing the fracture plate to the lateral surface of the ilium by applying screws through multiple screw holes on each side of the fracture.

According to an aspect of the present disclosure, a method for stabilizing a fracture of the ilium is provided. The method comprises: positioning a single fracture plate on a lateral surface of the ilium straddling the fracture such that at least three screw holes divided between two rows are on either side of the fracture; and screwing the fracture plate to the lateral surface of the ilium by applying screws through at least three screw holes on each side of the fracture.

According to an aspect of the present disclosure, an ilial fracture stabilization apparatus is provided, comprising at least one screw and means for stabilizing the ilial fracture.

### BRIEF DESCRIPTION OF THE DRAWINGS

Various aspects and embodiments will be described with reference to the following figures. It should be appreciated that the figures are not necessarily drawn to scale. Items appearing in multiple figures are indicated by the same reference number in all the figures in which they appear.

FIG. 1 illustrates an ilial fracture stabilization apparatus including an ilial fracture plate according to an embodiment of the present disclosure.

FIG. 2 is a perspective view of the ilial fracture plate of FIG. 1.

FIG. 3 illustrates an ilial fracture stabilization apparatus including an ilial fracture plate having ten holes and a variable width, according to an embodiment of the present disclosure.

FIG. 4 illustrates an ilial fracture stabilization apparatus including an ilial fracture plate having eleven holes and a variable width, according to an embodiment of the present disclosure.

FIG. 5 illustrates an ilial fracture stabilization apparatus including an ilial fracture plate having six holes and a variable width, according to an embodiment of the present disclosure.

FIG. 6 illustrates an ilial fracture stabilization apparatus including an ilial fracture plate having eight holes and a constant width, according to an embodiment of the present disclosure.

FIG. 7 illustrates an ilial fracture stabilization apparatus including an ilial fracture plate having seven holes, including figure-eight shaped holes, and a variable width, according to an embodiment of the present disclosure.

FIG. 8 is a coronal (frontal/dorsal) view of a whole pelvis with an ilial fracture stabilization apparatus secured to a lateral surface of the ilium, according to an embodiment of the present disclosure.

FIG. 9 is a top view of an ilial fracture plate having eight holes and a constant width, according to an embodiment of the present disclosure.

FIG. 10 is a side view of an ilial fracture stabilization apparatus having a planar ilial fracture plate and a plurality of screws, according to an embodiment of the present disclosure.

FIG. 11 is a side view of an ilial fracture stabilization apparatus having a curved ilial fracture plate and a plurality of screws, according to an embodiment of the present disclosure.

FIG. 12 is a flowchart of a method of securing an ilial fracture plate to a lateral surface of an ilium, according to an embodiment of the present disclosure.

### DETAILED DESCRIPTION

Aspects of the present disclosure provide methods and apparatuses for stabilization of ilial fractures in animals. The apparatuses may include a fracture plate having multiple rows of screw holes positioned to allow for securing, on both sides of the fracture, multiple screws in close proximity to the fracture. The methods may involve securing a fracture plate to an ilium such that the fracture plate straddles the fracture, and securing multiple screws through the fracture plate in close proximity to the fracture on both sides of the facture including in a dorsal to ventral orientation.

Ilial fractures are common injuries in animals, including, but not limited to, dogs and cats. The most common orientation of such fractures is oblique from cranioventral (anterior ventral) to caudodorsal (posterior dorsal), meaning the fracture is angled across the ilium between a higher, rearward point (nearer the animal's tail) and a lower, forward point (nearer the animal's head) on the ilium. Such fractures typically do not run along the shortest path from the top to bottom of the ilium.

As a result of the typical oblique orientation of ilial fractures, the area of the ilium on either side of the fracture that is usable for securing a stabilization device by screws or otherwise may be limited. On one side of the fracture, the usable space is limited by the distance between the fracture and the acetabulum (hip joint). A stabilization device should not interfere with the acetabulum. On the other side of the fracture, the usable space is limited by the distance from the fracture to the ilial wing, and, pertinently, the amount of dense cortical bone between the fracture and the ilial wing. This is because the ilial wing is typically thinner and less dense than other portions of the ilium, and therefore less suitable for holding screws. In practice, the distance from the oblique fracture to the acetabulum and/or from the fracture to the ilial wing is no more than a few centimeters, depending on the size of the animal.

In addition, fractures of the ilium can and should generally be treated without repositioning of the acetabulum. Repositioning (ventroversion) of the acetabulum is performed in animals with hip dysplasia or other defects when performing a double or triple pelvic osteotomy. With a double or triple pelvic osteotomy, a cut (osteotomy) is made in the ilium and an angulation or step purposefully introduced between the two portions of the ilium with an osteotomy plate that exhibits a step or discontinuity between two sides of the plate. Such repositioning (ventroversion) is problematic in the case of treating a fracture of the ilium.

Aspects of the present disclosure provide an ilial fracture plate with multiple rows of screw holes positioned to allow for securing, on both sides of a fracture of the ilium, multiple screws in close proximity to the fracture. The multiple rows of screw holes facilitate placement of multiple screws in close proximity to the fracture on both sides of the fracture, in dense bone suitable for receiving and retaining such screws. Also, the screw holes can be positioned from cranial (anterior) to caudal (posterior) as well as dorsally (superior) and ventrally (inferior). The screw holes may be locking or non-locking. The screw holes may take various shapes. In some embodiments, at least some of the screw holes are compression screw holes, providing compression of the fracture when the fracture plate is secured. Also, by providing multiple rows of screw holes in the ilial fracture plate, a sufficient number of screws may be fitted without necessarily filling all the screw holes. That is, some of the screw holes may be left open.

The use of a fracture plate with multiple rows of screw holes differs from use of a fracture plate with a single row of screw holes, and from use of multiple single-row fracture plates in combination. A single row fracture plate, sometimes referred to as a “straight plate,” has a single row of screw holes such that the holes are positioned at increasing distance from the fracture. Given the limitations on useful available bone for securing a fracture plate to the ilium, as described above, use of a single row fracture plate is unsatisfactory for securing a sufficient number (e.g., three or more) of screws on either side of the fracture in dense bone without interfering with the acetabulum posteriorly, or without placing screws in the thin bone anterior to the fracture and in the region of the ilial wing. In contrast to using multiple straight plates together, use of a single multiple-row fracture plate as described herein provides greater stability and case of use, among other potential benefits. The inventor has appreciated that a single plate having multiple rows of screw holes provides greater strength, and thus improved stability for repair of the fractured bone. As a result, better patient outcomes are achieved.

According to an aspect of the present disclosure, a method of stabilizing an ilial fracture is provided. The method comprises securing a single multiple-row fracture plate to the ilium. The fracture plate has multiple rows of screw holes such that multiple screws can be positioned in close proximity to the fracture on either side of the fracture. The method comprises securing multiple screws on both sides of the fracture, with multiple screws on both sides of the fracture being in close proximity to the fracture. In some embodiments, two screws on either side of the fracture are substantially equidistant from the fracture. In some embodiments, the method comprises securing a screw through a compression hole, thereby applying compression to the fracture.

According to an aspect of the present disclosure, means for stabilizing an ilial fracture are provided. In some embodiments, the means comprise a single plate body having multiple rows of screw holes. The multiple rows of screw holes are positioned within the plate body such that multiple screw holes will be within close proximity to the fracture on either side of the fracture.

The aspects and embodiments described above, as well as additional aspects and embodiments, are described further below. These aspects and/or embodiments may be used individually, all together, or in any combination of two or more, as the disclosure is not limited in this respect.

According to an aspect of the present disclosure, an ilial fracture stabilization apparatus comprises a planar or curved fracture plate having multiple rows of screw holes. The multiple rows of screw holes provide a plurality of screw holes configured to be positioned in close proximity to an ilial fracture on either side of the fracture. The fracture plate has a width sufficient to accommodate the multiple (e.g., two) rows of screw holes. Thus, in at least some embodiments, the fracture plate has a width that is greater than the combined width of two adjacent screw holes. In some embodiments, the facture plate is curved.

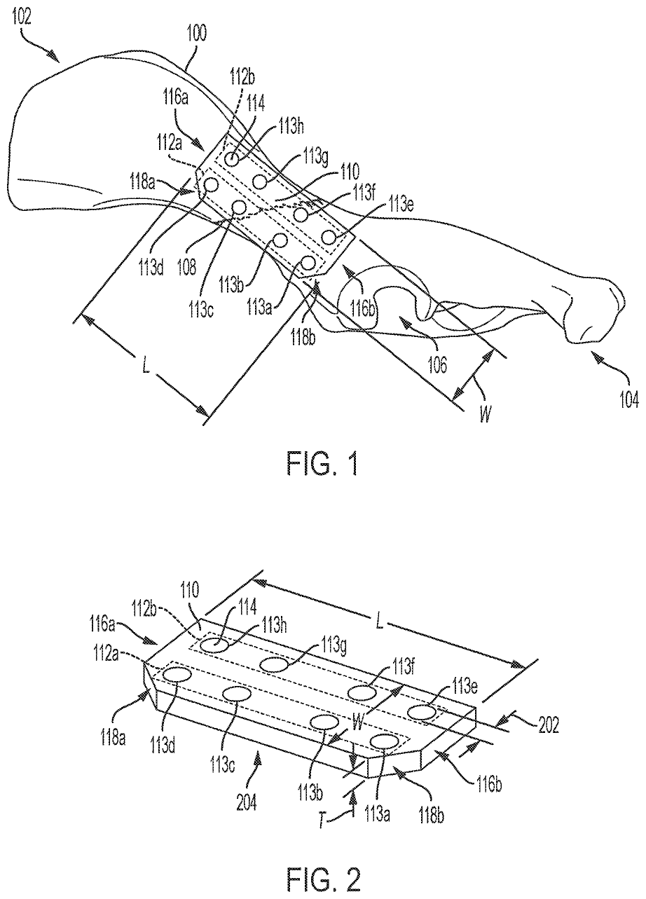

FIG. 1 illustrates an ilial fracture stabilization apparatus including an ilial fracture plate according to an embodiment of the present disclosure. The ilium 100 has a wing 102. The illustrated bone also includes the ischium 104 and acetabulum 106. The ilium 100 is shown to have a fracture 108. The ilial fracture plate 110 has multiple rows of screw holes, including a first row 112a and second row 112b of holes 114.

In this example, the fracture 108 is an oblique fracture, angled from cranioventral (anterior ventral) to caudodorsal (posterior dorsal). The same is true of the other examples described herein comprising the fracture 108. However, the ilial fracture plates described are not limited to use with fractures of any particular orientation, and thus the fracture 108 represents a non-limiting example.

The ilial fracture plate 110 is a multi-row fracture plate. In this example, the ilial fracture plate 110 includes two rows—first row 112a and second row 112b—of holes 114. The rows are oriented along the length L of the ilial fracture plate 110. In this example, the rows 112a and 112b of holes 114 are substantially perpendicular to the width W of the ilial fracture plate 110. Including two rows of holes 114 in the ilial fracture plate provides increased ability to have multiple screws in close proximity to the fracture on either side of the fracture, without requiring the plate to be so wide as to make it impractical to use. For example, the holes 114 and therefore any corresponding screws may each be within approximately 8 cm of the fracture, within 7 cm of the fracture, within 6 cm of the fracture, or within 5 cm of the fracture in some embodiments, or closer, including to as little as 0.2 cm (e.g., each of the screws may be between 0.2 cm and 6 cm from the fracture in some embodiments). In some embodiments more than two rows may be included, particularly if the size of the screw holes is sufficiently small to allow for the width of the plate—described further below—to fit well on the ilium.

The rows of holes 114 of the ilial fracture plate may be aligned in various ways. In some embodiments, the rows of holes 114 of an ilial fracture plate are parallel or substantially parallel. FIG. 1 is an example of such alignment of the rows of holes 114. First row 112a and second row 112b are substantially parallel. Not all embodiments are limited in this respect, however. For example, the rows of holes 114 may be angled relative to each other in some embodiments. Alternatively, the holes 114 may include a hole that is not in-line with the rows. For instance, one of the holes of one of the rows may be offset from the other holes in that row, such that the rows are not parallel.

The two rows 112a and 112b provide a total of eight holes 114, such that the ilial fracture plate 110 may be considered an 8-hole fracture plate. As described above, the multi-row nature of the ilial fracture plate 110 allows for multiple screws to be positioned in close proximity to the fracture 108 on either side of the fracture 108. In the example of FIG. 1, first row 112a comprises holes 113a, 113b, 113c, and 113d. Second row 112b comprises holes 113c. 113f, 113g, and 113h. In the illustrated configuration, four holes 113b, 113c, 113f, and 113g are all adjacent the fracture 108. Holes 113a, 113d, 113e, and 113h are further from the fracture 108, but still in close proximity. Thus, in contrast to a single row plate which may allow for only one or two holes in close proximity to an ilial fracture on either side of the fracture, a multi-row ilial fracture plate may allow for three, four, or more holes in close proximity to the fracture on either side of the fracture. In addition, some of the holes may be left open, and still a sufficient number of screws fitted in the other holes to provide a secure attachment of the ilial fracture plate 110 to the ilium 100. For example, if the hole 113c is left open (e.g., if the fracture 108 ran underneath that hole), there still is a sufficient number of holes 113d, 113g, and 113h on the anterior side of the fracture 108 to allow for securing the ilial fracture plate 110 to the ilium with the screws being in solid bone. The same is true of other embodiments described herein.

It should be appreciated by reference to FIG. 1 that the holes 114 of the ilial fracture plate 110 allow for positioning of screws from cranial (anterior) to caudal (posterior) as well as dorsally (superior) and ventrally (inferior). Such a configuration facilitates stabilization of the fracture site with the ilial fracture plate 110.

As described above, the ilial fracture plate 110 includes eight holes 104. However, other numbers of holes may be included, examples of which are illustrated in other drawings herein. For example, each row may include at least three holes in some embodiments. Ilial fracture plates according to embodiments of the present disclosure may include a total of 6-12 holes. Still other numbers are possible.

The holes 114 may be of various shapes and types. In some embodiments, the holes are circular. In some embodiments, the holes are oval. Snowman-shaped or figure-eight shaped holes may be used. The holes may be compression holes, providing the ability to compress the fracture 108. For instance, oval holes with compression capability may be used. Holes that provide a combination of locking and non-locking functionality may be used. In some embodiments, small holes for wire placement for temporary stabilization of the ilial fracture plate 110 to the ilium 100 may be used. Any combination of the types of holes just described may be used.

The holes 114 may be of various sizes to accommodate different sized animals, such as different sized dogs. For example, screws ranging from 1.1 mm to 4.5 mm in diameter, including any value within that range, may be used to secure the ilial fracture plate 110. Thus, the holes may be sized accordingly. In some embodiments, the size of the holes 114 substantially matches the size of the screw used, such as when a locking screw is used. In some embodiments, the size of the holes 114 is larger than the size of the screws, such as when the hole is an oval hole that offers the ability to compress the fracture and the screw to be used in that hole is a non-locking screw. Thus, the size of the holes may range from approximately 1.1 mm to approximately 5 mm in some embodiments, including any value within that range. The holes 114 need not all be the same size. In some embodiments, different size holes are provided in the ilial fracture plate 110.

Various types of screws may be used with the holes 114. In some embodiments, locking screws may be used. In other embodiments, non-locking screws may be used. In some embodiments, a combination of locking and non-locking screws may be used. Consequently, the holes 114 will also vary according to which type of screws are to be used in those holes. In some embodiments, the screws are angle-stable screws, and may use a suitable press fit mechanism or be threaded. In some embodiments in which angle-stable screws are used, the angle-stable screws are parallel to each other. In some embodiments in which angle-stable screws are used, the angle-stable screws may be non-parallel (e.g., divergent or convergent). In some embodiments in which angle-stable screws are used, some of the angle-stable screws may be parallel each other and others may be non-parallel each other.

The ilial fracture plate 110 has a length L, width W, and thickness T. The thickness T is shown in FIG. 2, which is a perspective view of the ilial fracture plate 110.

The length L, width W, and thickness T may be selected to provide desired characteristics of the ilial fracture plate 110. The length L is greater than the width W in this embodiment, and generally in the examples described herein. The length L may be between 2-14 cm in some non-limiting embodiments, including any value or range of values within that range. The width W may be sufficient to accommodate at least two rows of holes 114. In the example of FIG. 1, the width W is sufficient to accommodate the first row 112a and second row 112b of holes 114. As shown in FIG. 2, the holes 114 may have a width 202. The width 202 is the size of the hole in the direction of the width W. If the holes 114 are circles, then the width 202 is a diameter. However, since other shapes are possible for holes 114, as described further below, the width 202 is not a diameter in all embodiments. Thus, the width W may be at least twice as large as the width 202 to accommodate the two rows of holes 114. In practice, the width W will be between 1.5× and 5× the combined width of the holes 114 in the direction of the width W. That is, in the example of FIG. 2, the width W will be between 1.5× and 7× the combined width of hole 113c and hole 113g, that combined width in this non-limiting example being twice the width 202 shown in FIG. 2. In some embodiments, the width W will be between 2× and 5× the combined width of hole 113c and hole 113g. In some embodiments, the width W is between 1 cm and 5 cm. The thickness T of the ililal fracture plate 110 is selected to be sufficient to provide stability for the patient when implanted and to withstand the forces upon the joined section of bone. In some embodiments, the thickness Tis between 0.2 cm and 1 cm.

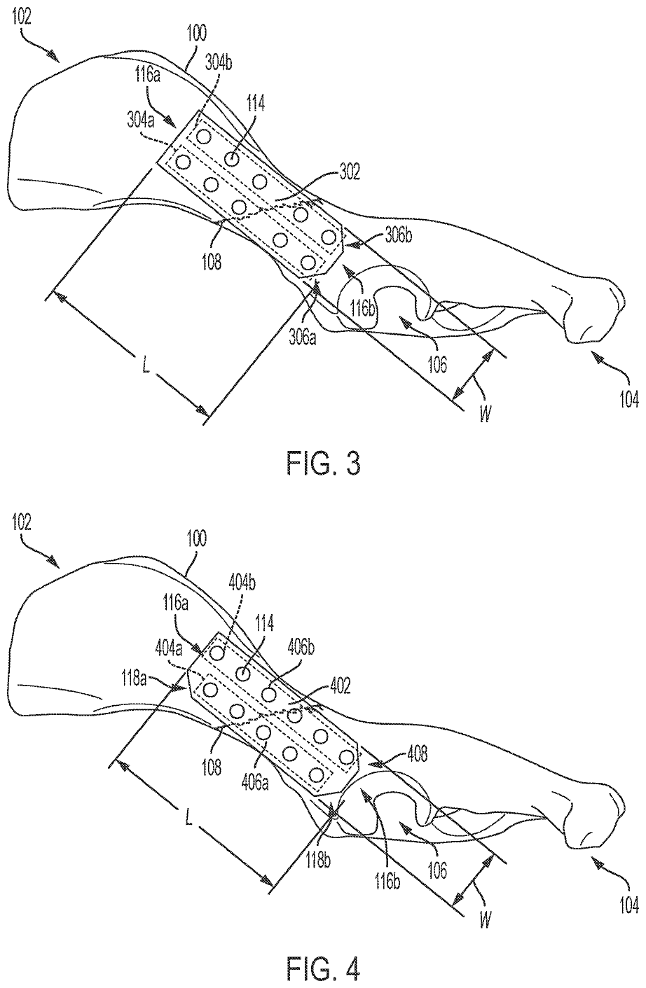

The width W of the ilial fracture plate 110 may be constant or varied along the length L. Referring to FIG. 1, the ilial fracture plate 110 includes a first end 116a and a second end 116b. The width W is variable along the length between the first end 116a and second end 116b. As shown, the ilial fracture plate 110 has two modified edges. Specifically, a first angled edge 118a is positioned at the first end 116a, and a second angled edge 118b is positioned at the second end 116b. As a result, the width W is narrower at the first end 116a and second end 116b than at a midpoint along the length L. That is, the ilial fracture plate 110 narrows at the two ends 116a and 116b. The amount of reduction in width may be selected in some embodiments to facilitate placement of the ilial fracture plate without interfering with the acetabulum 106. For instance, as shown, the second end 116b may be positioned proximate (or craniodorsal/anterior-superior to) the acetabulum. Including the second angled edge 118b may allow positioning the ilial fracture plate 110 appropriately with respect to the fracture 108 without the ilial fracture plate 110 interfering with the acetabulum 106. A greater degree of narrowing of the width W at the second end 116b may facilitate such positioning. It should be appreciated from the foregoing description of first angled edge 118a and second angled edge 118b that in some embodiments the ilial fracture plate may have modified edges. FIGS. 3, 4, 5, and 7 illustrate other embodiments of ilial fracture plates with modified edges.

Other embodiments described herein also include a variable width W of the ilial fracture plate. The width may narrow at one or both ends of the ilial fracture plate in some embodiments. The width may narrow from a single angled edge (e.g., as in FIGS. 1, 4, 5, and 7), or two angled edges at the end (e.g., FIGS. 3 and 4).

Referring again to FIG. 2, the ilial fracture plate 110 includes a bottom surface 204 configured to contact the ilium 100 when secured to the ilium 100. The bottom surface 204 may be smooth or contoured. A smooth surface provides greater contact area to the ilium. A contoured surface provides reduced contact area, which may be desired in some instances, for instance to lessen the impact of the fracture plate on microvasculature. Examples of contoured bottom surfaces include scalloped surfaces.

The ilial fracture plate 110 may be made of any suitable biocompatible material or combination of materials. In some embodiments, the ilial fracture plate 110 is made of titanium. In other embodiments, the ilial fracture plate 110 is made of titanium alloy, stainless steel, cobalt-based alloys such as, but not limited to, cobalt chrome, polymers, or ceramics.

The ilial fracture plate 110 is shown as being planar, or substantially planar. However, alternative configurations are provided. The ilium has a natural curve. In some embodiments, the ilial fracture plates of the types described herein exhibit a curvature or contour facilitating mating of the plate to a lateral surface of the ilium. The degree of curvature is selected to accommodate the natural curvature of the ilium. In some embodiments, the ilial fracture plate exhibits a radius of curvature between 5 degrees and 30 degrees, in some embodiments between 8 degrees and 20 degrees, in some embodiments between 10 degrees and 15 degrees, or any value or range of values within such ranges. An example is described further below in the context of FIG. 11.

FIG. 3 illustrates an ilial fracture stabilization apparatus including an ilial fracture plate having ten holes and a variable width, according to an embodiment of the present disclosure. The ilial fracture plate 302 has a first row 304a and a second row 304b each having five holes 114. The ilial fracture plate 302 has a width W that narrows at the second end 116b, owing to two angled edges 306a and 306b. The two angled edges 306a and 306b are angled inwardly. As illustrated, the narrower end of ilial fracture plate 302 is therefore proximate the acetabulum 106.

FIG. 4 illustrates an ilial fracture stabilization apparatus including an ilial fracture plate having eleven holes and a variable width, according to an embodiment of the present disclosure. The ilial fracture plate 402 comprises a first row 404a and second row 404b of holes 114. The first row 404a comprises five holes 114. The second row 404b comprises six holes 114. In this embodiment, the hole placement is asymmetric. The third hole 406a of the first row 404a is offset along the length L relative to the third hole 406b of the second row 404b. The same is true of the other respective holes of the two rows 404a and 404b. Asymmetric placement of the holes 114 may facilitate positioning of the ilial fracture plate relative to the fracture 108. For instance, when the fracture 108 is oblique, as illustrated in the example of FIG. 4, an asymmetric placement of the holes 114 may facilitate positioning of the ilial fracture plate 402 without any of the holes being positioned directly over the fracture 108. Thus, a sufficient number of holes—and therefore screws placed in the holes—may be achieved on both sides of the fracture 108 despite the oblique nature of the fracture, with the holes being in close proximity to the fracture. The same is true for other embodiments described herein as having asymmetric placement of the screw holes.

The ilial fracture plate 402 comprises the first angled edge 118a and second angled edge 118b, and also third angled edge 408. These are modified edges. Thus, the width W is narrower at the first end 116a than at a midpoint along the length L, and the width W is narrower at the second end 116b than at the first end 116a in the example of FIG. 4.

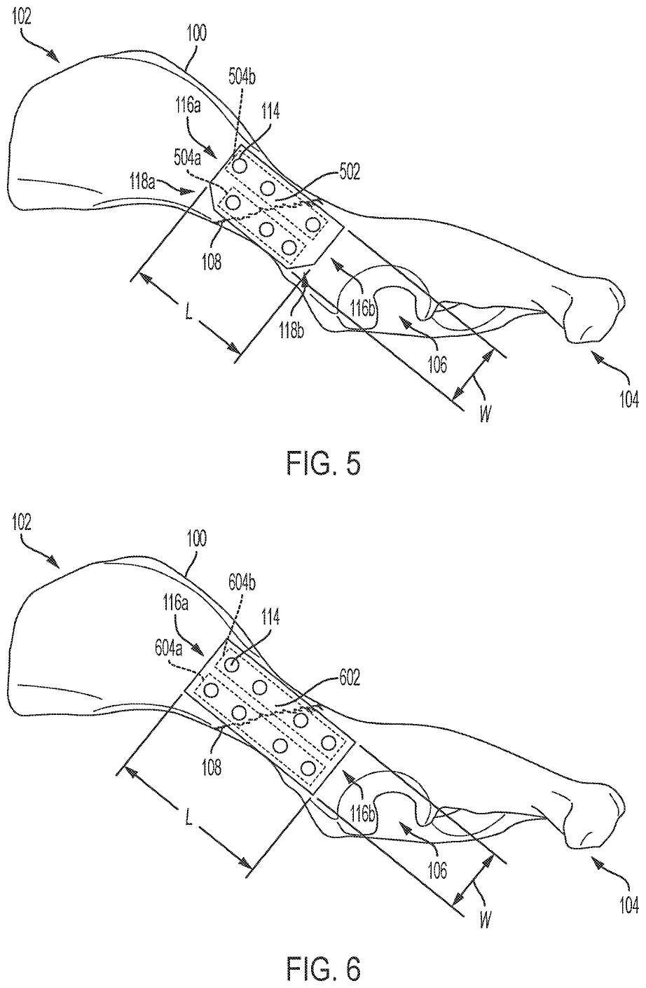

FIG. 5 illustrates an ilial fracture stabilization apparatus including an ilial fracture plate having six holes and a variable width, according to an embodiment of the present disclosure. The ilial fracture plate 502 comprises a first row 504a and second row 504b of holes 114, each row having three holes in this non-limiting example. As illustrated, the placement of holes 114 is asymmetric. The positioning of the holes 114 of the first row 504a along the length L differs from the placement of respective holes 114 of the second row 504b along the length L. Thus, as illustrated, the ilial fracture plate 502 may be positioned relative to the fracture 108 such that the holes 114 on either side of the fracture 108 form a triangle. In this configuration, three screws may be placed on either side of the fracture 108, providing for a secure attachment of the ilial fracture plate 502 to the ilium 100. In the embodiment of FIG. 5, the ilial fracture plate 502 narrows at the first end 116a and the second end 116b owing to the first angled edge 118a and second angled edge 118b.

FIG. 6 illustrates an ilial fracture stabilization apparatus including an ilial fracture plate having eight holes and a constant width, according to an embodiment of the present disclosure. The ilial fracture plate 602 comprises a first row 604a and a second row 604b of holes 114. The two rows 604a and 604b have an equal number of holes, in this case four holes 114 per row. The holes are placed symmetrically. The width W of the plate 602 is constant along its length L.

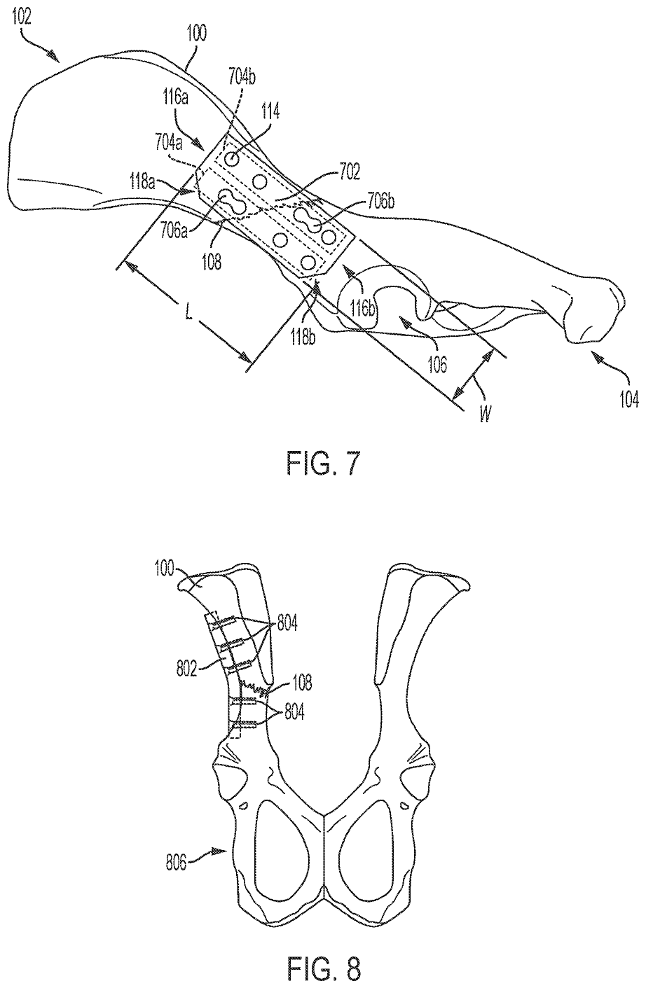

FIG. 7 illustrates an ilial fracture stabilization apparatus including an ilial fracture plate having seven holes, including figure-eight shaped holes, and a variable width, according to an embodiment of the present disclosure. The ilial fracture plate 702 comprises a first row 704a and second row 704b of holes 114. In this embodiment, the first row 704a comprises three holes and the second row 704b comprises four holes. Each row includes a figure-eight shaped hole. Specifically, row 704a includes figure-eight shaped hole 706a and row 704b includes figure-eight shaped hole 706b. The figure-eight shaped holes 706a and 706b may provide a combination of locking and non-locking features.

FIG. 8 is a coronal (frontal/dorsal) view of a whole pelvis with an ilial stabilization apparatus secured to a lateral surface of the ilium, according to an embodiment of the present disclosure. The pelvis 806 includes the ilium 100, now shown in a front view. An ilial fracture plate 802 is shown in a side view, and is secured to the lateral surface of the ilium 100 such that the screws 804 are screwed into the bone. The ilial fracture plate 802 straddles the fracture 108. In this non-limiting example, a single row of screws 804 is shown owing to the side orientation of the ilial fracture plate 802. Three of the screws 804 of the illustrated row are on one side of the fracture 108 and two screws of the illustrated row are on the other side of the fracture 108. Because the ilial fracture plate 802 comprises additional rows of holes not visible in this view, more than two screws may be secured on either side of the fracture 108. In this example, the ilial fracture plate 802 is shown as having a curvature to align with the lateral surface of the ilium 100.

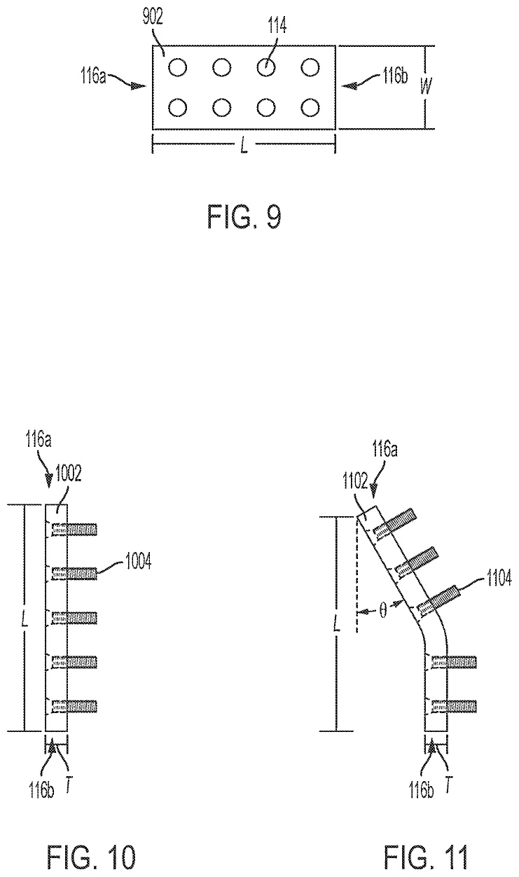

FIG. 9 is a top view of an ilial fracture plate 902 having eight holes 114, a length L, and a constant width W, according to an embodiment of the present disclosure. The holes 114 of the ilial fracture plate 902 are circular holes. However, other types of holes of the types described herein may additionally or alternatively be used in place of one of the illustrated holes.

FIG. 10 is a side view of an ilial stabilization apparatus having a flat planar ilial fracture plate and a plurality of screws, according to an embodiment of the present disclosure. The ilial fracture plate 1002 has a first end 116a and second end 116b. The ilial fracture plate 1002 has a length L and thickness T, assuming any of the values described herein for such dimensions. The ilial fracture plate 1002 supports up to five screws 1004 per row in the non-limiting example of FIG. 10. In this figure, only a single row of screws 1004 is visible due to the orientation of the ilial fracture plate 1002. The ilial fracture plate has multiple rows of screw holes of the types described previously herein.

The ilial fracture plate 1002 is flat in the illustrated example. The ilial fracture plate 1002 may be secured to the ilium of an animal in such a configuration or may be bent or contoured prior to application to the animal. When secured in a flat state, there may be no angular offset between the screws 1004 and application of the fracture plate to the ilium may avoid any re-alignment or ventroversion of the acetabulum. However, as described above, in some embodiments a flat fracture plate may bent or contoured. For example, the flat ilial fracture plate may be bent to have a curvature substantially matching that of the patient's ilium. Such bending may be accomplished in any suitable manner.

FIG. 11 is a side view of an ilial stabilization apparatus having a curved ilial fracture plate and a plurality of screws, according to an embodiment of the present disclosure. As described above, some embodiments of an ilial fracture plate exhibit a curvature facilitating placement of the ilial fracture plate against a lateral surface of the ilium. Different animals exhibit a different degree of curvature of the ilium. Thus, ilial fracture plates with different degrees of curvature may be provided. The curvature may be introduced during manufacture, such that the ilial fracture plate may be considered to be pre-contoured. The ilial fracture plate 1102 supports up to five screws 1104 per row in the non-limiting example of FIG. 11. In this figure, only a single row of screws 1104 is visible due to the orientation of the ilial fracture plate 1102. The ilial fracture plate 1102 has a first end 116a and second end 116b, and exhibits the length L, thickness T, and curvature θ. The degree of curvature θ may assume any of the values for such curvature described herein (e.g., between 5 degrees and 30 degrees, including any value in that range). The curvature may be gradual in some embodiments or may be more abrupt, as shown.

According to an embodiment of the present disclosure, a single multi-row ilial fracture plate is provided. The single multi-row ilial fracture plate comprises two rows of screw holes. Each of the two rows comprises at least four screw holes and each row comprises at least one compression hole. The single multi-row ilial fracture plate has a width that varies along its length, such that the width is narrower at one end of the ilial fracture plate than at the other. The narrower end of the single multi-row ilial fracture plate is configured to be positioned proximate the acetabulum when secured to a lateral surface of the ilium. The single multi-row ilial fracture plate exhibits a curvature between 5 degrees and 30 degrees. The single multi-row ilial fracture plate is made of titanium or a titanium alloy. The single multi-row lilial fracture plate may have suitable dimensions, including a length between 2-14 cm, a width between 1-5 cm, and a thickness between 0.2 cm and 1 cm.

According to an aspect of the present disclosure, a method of stabilizing an ilial fracture is provided. The method comprises securing a single multiple-row fracture plate to the ilium. The fracture plate has multiple rows of screw holes such that multiple screws can be positioned in close proximity to the fracture on either side of the fracture. The method comprises securing multiple screws on both sides of the fracture, with multiple screws on both sides of the fracture being in close proximity to the fracture (e.g., within 7 cm of the fracture, within 5 cm of the fracture, within 2 cm of the fracture, or less, including down to 0.2 cm or less in some embodiments). In some embodiments, multiple screws on either side of the fracture are substantially equidistant from the fracture. In some embodiments, the method comprises securing a screw through a compression hole, thereby applying compression to the fracture.

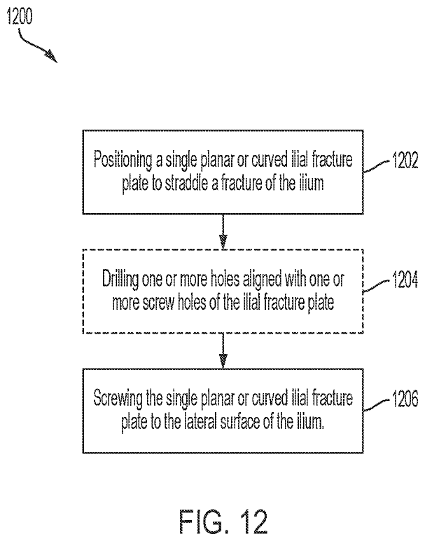

FIG. 12 is a flowchart of a method of securing an ilial fracture plate to a lateral surface of an ilium, according to an embodiment of the present disclosure. The method 1200 begins at stage 1202 with positioning a single planar or curved ilial fracture plate such that it straddles a fracture of the ilium. The ilial fracture plate may be any of the types of single plates described herein, such as those illustrated in FIGS. 1-11. Positioning the single planar or curved ilial fracture plate to straddle the fracture may involve positioning the single planar or curved ilial fracture plate such that multiple screw holes of the plate are disposed on either side of the fracture in close proximity to the fracture. In some embodiments, at least three screw holes are positioned on both sides of the fracture.

As described above, ilial fracture plates of the types described herein may take various forms. In some embodiments, the single planar or curved ilial fracture plate has a first end, a second end, a length from the first end to the second end, and a width that varies along the length such that the width narrows at the first end. For example, the ilial fracture plate of FIG. 1 is such an example. Stage 1202 of method 1200 may involve positioning the single fracture plate with the first end proximate an acetabulum. For example, as shown in FIGS. 1, 3, 4, 5, and 7, the ilial fracture plate may be positioned such that a narrowed end of the ilial fracture plate is proximate the acetabulum.

The method 1200 optionally involves drilling one or more holes in the ilium at stage 1204. The optional nature of this stage is reflected by the dashing in FIG. 12. Drilling one or more holes in the ilium may involve drilling one or more holes aligned with one or more screw holes of the single planar or curved ilial fracture plate. Drilling such holes in the ilium may facilitate subsequent insertion of a screw.

The method proceeds to stage 1206, which involves screwing the single planar or curved ilial fracture plate to the lateral surface of the ilium. Since the positioning at stage 1202 may involve positioning multiple screw holes on either side of the fracture, stage 1206 may involve securing multiple screws on either side of the fracture. In some embodiments, three or more screws may be secured on each side of the fracture, within close proximity to the fracture.

Since the ilial fracture plates described herein may include various types of screw holes, stage 1206 may involve using various types of screws. In some embodiments, stage 1206 involves applying a screw through at least one compression hole of the ilial fracture plate.

According to an aspect of the present disclosure, a method for stabilizing a fracture of the ilium is provided. The method comprises: positioning a single fracture plate on a lateral surface of the ilium straddling the fracture such that at least three screw holes divided between two rows are on either side of the fracture; and screwing the fracture plate to the lateral surface of the ilium by applying screws through at least three screw holes on each side of the fracture.

In some embodiments, screwing the fracture plate to the lateral surface of the ilium comprises applying a screw through at least one compression hole of the fracture plate.

In some embodiments, the method further comprises drilling a hole in the ilium aligned with a first screw hole of the fracture plate. Screwing the fracture plate to the lateral surface of the ilium comprises applying a screw to the hole in the ilium.

In some embodiments, the single fracture plate has a first end, a second end, a length from the first end to the second end, and a width that varies along the length such that the width narrows at the first end, and wherein positioning the single fracture plate on the lateral surface of the ilium comprises positioning the single fracture plate with the first end proximate an acetabulum.

According to an aspect of the present application, an apparatus for stabilization of ilial fractures is provided. The apparatus comprises a planar or curved fracture plate having a first end and second end, and two rows of screw holes positioned between the first end and second end. Each of the two rows of screw holes may comprise three or more screw holes and a total number of screw holes of the two rows of screw holes may be between six and twelve. The planar or curved fracture plate has a length, a width, and a thickness, and the width of the planar or curved fracture plate may be greater than a combined width of two adjacent screw holes along a dimension of the width. The width may be variable along the length such that the width is narrower at the first end than at a midpoint of the planar or curved fracture plate.

In some embodiments, the width is narrower at the second end than at the midpoint of the planar curved fracture plate.

In some embodiments, at least one hole of a first row of the two rows of screw holes is oval. In some embodiments, at least one hole of a first row of the two rows is figure-eight shaped. In some embodiments, the planar or curved fracture plates comprises both oval and figure-eight shaped holes. In some embodiments, the two rows of screw holes are placed asymmetrically. The thickness of the fracture plate may be 1 cm or less and the length may be 14 cm or less.

In some embodiments, at least one hole of a first row of the two rows of screw holes is figure-eight shaped.

In some embodiments, the planar or curved fracture plate is curved. The degree of curvature may be between five degrees and thirty degrees.

In some embodiments, the two rows of screw holes are placed asymmetrically.

According to an aspect of the disclosure, an apparatus for stabilization of ilial fractures is provided, comprising a planar or curved fracture plate comprising multiple rows of screw holes. The planar or curved fracture plate has a length, a width, and a thickness. The width of the fracture plate is greater than a combined opening size of two screw holes along a dimension of the width.

In some embodiments, the planar or curved fracture plate is curved, and in some such embodiments the degree of curvature is between 5 degrees and 30 degrees. In some embodiments, the planar or curved fracture plate is substantially planar.

In some embodiments, the multiple rows of screw holes comprise at least one compression hole. In some such embodiments, the multiple rows of screw holes comprise at least one compression hole in each row.

In some embodiments, the multiple rows of screw holes are substantially parallel to each other.

In some embodiments, the multiple rows of screw holes comprise two holes on each side of a centerline bisecting the fracture plate along the length of the fracture plate.

In some embodiments, the planar or curved fracture plate has a first end and a second end, and the width is variable along the length such that the width narrows at the first end and/or second end of the fracture plate. In some such embodiments, the width narrows at the first end, and the first end is configured to be positioned proximate an acetabulum.

In some embodiments, the planar or curved fracture plate is curved, the curved fracture plate has a first end and a second end, and the width is variable along the length such that the width narrows at the first end and not the second end.

In some such embodiments, the multiple rows of screw holes comprise an asymmetric arrangement of screw holes.

In some embodiments, the multiple rows of screw holes comprise at least one compression screw hole.

In some embodiments, the multiple rows of screw holes comprise a plurality of screw holes including at least one locking screw hole.

In some embodiments, the planar or curved fracture plate does not exhibit angulation.

In some embodiments, the planar or curved fracture plate does not exhibit a discontinuity.

Variations on the embodiments described thus far are possible. For example, the number of holes provided with a multi-row ilial fracture plate may range from four to twelve holes, or more. The holes may be evenly dispersed with respect to a centerline of the plate (a midpoint of the length of the plate) or may be distributed with more holes closer to one end of the ilial fracture plate than the other end.

Aspects of the present disclosure provide various benefits, some of which have been described already herein. Some examples are now listed. It should be appreciated that benefits other than those now listed are possible, and that not all embodiments provide all, or even any, of the benefits listed here.

Some aspects provide ilial fracture stabilization systems allowing for case of positioning and securing to a fractured ilium. For example, working with a single ilial fracture plate simplifies positioning compared to trying to position multiple separate fracture plates. Also, ilial fracture plates with multiple rows of screw holes provide greater flexibility in selecting which screw holes to use and which (if any) to leave open, while still providing a sufficient number of screws to sufficiently secure the fracture plate to the bone. Some aspects provide greater stability of the fracture site, thus enhancing healing and improving patient outcomes. Some aspects allow for placement of multiple screws in solid (dense cortical) bone on both sides of the fracture of the ilium, irrespective of whether the fracture is posterior in the ilium, mid-body in the ilium, or in the anterior portion of the ilium. Such screws may be positioned without interfering with either the hip joint posteriorly or being placed in the soft, thin bone of the ilial wing anteriorly. Some aspects provide for stabilization of an ilial fracture without introducing an angulation or discontinuity in the ilium and without repositioning the acetabulum at all or to any meaningful degree. None of the illustrated examples of multi-row ilial fracture plates has an angulation or discontinuity that would result in acetabular ventroversion as is obtained when performing a double or triple pelvic osteotomy.

Some embodiments may provide lower cost for ilial fracture stabilization procedures, owing to the use of a single plate.

Some aspects of the technology provide an improved method and apparatus for stabilization of fractures of the ilium in non-human animals. The apparatus may include an ilial fixation device (e.g., a fracture plate) allowing for placement of numerous screws in solid bone on both sides of the fracture, whether the fracture is posterior in the ilium, mid-ilium, or in the anterior portion of the ilium. According to some embodiments the ilial fixation device includes multiple rows of screw holes. Consequently, numerous screws can be placed close to the fracture side and in solid bone while reducing interference with the hip joint posteriorly or reliance on screws placed in the soft bone of the ilial wing anteriorly.

Various inventive aspects may be embodied as one or more processes, of which an example has been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Accordingly, the foregoing description and drawings are by way of example only.

1. An apparatus for stabilization of ilial fractures, comprising:
a curved fracture plate having a first end and second end, and comprising two rows of screw holes positioned between the first end and second end,
wherein each of the two rows of screw holes comprises three or more screw holes and a total number of screw holes of the two rows of screw holes is between six and twelve,
wherein the planar or curved fracture plate has a length, a width, and a thickness, and
wherein the width of the planar or curved fracture plate is greater than a combined width of two adjacent screw holes along a dimension of the width, and
wherein the width is variable along the length such that the width is narrower at the first end than at a midpoint of the planar or curved fracture plate.

2. The apparatus of claim 1, wherein the width is narrower at the second end than at the midpoint of the planar curved fracture plate.

3. The apparatus of claim 1, wherein at least one hole of a first row of the two rows of screw holes is oval.

4-5. (canceled)

6. The apparatus of claim 1, wherein the thickness is 1 cm or less and wherein the length is 14 cm or less.

7. The apparatus of claim 1, wherein at least one hole of a first row of the two rows of screw holes is figure-eight shaped.

8. (canceled)

9. The apparatus of claim 1, wherein the curved fracture plate has a degree of curvature between five degrees and thirty degrees.

10. The apparatus of claim 1, wherein the two rows of screw holes are placed asymmetrically.

11. An apparatus for stabilization of ilial fractures, comprising:
a planar fracture plate comprising multiple rows of screw holes,
wherein the planar fracture plate has a length, a width, and a thickness, and
wherein the width of the fracture plate is variable along the length of the fracture plate and, for at least some locations along the length of the fracture plate, the width is greater than a combined opening size of two screw holes in a dimension of the width.

12-14. (canceled)

15. The apparatus of claim 11, wherein the multiple rows of screw holes comprise at least one compression hole.

16. The apparatus of claim 15, wherein the multiple rows of screw holes comprise at least one compression hole in each row.

17. The apparatus of claim 11, wherein the multiple rows of screw holes are substantially parallel to each other.

18. (canceled)

19. The apparatus of claim 11, wherein the width narrows at the first end, and wherein the first end is configured to be positioned proximate an acetabulum.

20. (canceled)

21. The apparatus of claim 11, wherein the multiple rows of screw holes comprise an asymmetric arrangement of screw holes.

22. The apparatus of claim 21, wherein the multiple rows of screw holes comprise at least one compression screw hole.

23. The apparatus of claim 21, wherein the multiple rows of screw holes comprise a plurality of screw holes including at least one locking screw hole.

24. The apparatus of claim 11, wherein the planar fracture plate does not exhibit angulation.

25. The apparatus of claim 11, wherein the planar fracture plate does not exhibit a discontinuity.

26-30. (canceled)
